(19) United States
(12) Patent Application Publication
(10) Pub. No.: US 2026/0258076 A1
(43) Pub. Date: Sep. 3, 2026

(54) Process for the preparation of a fatty acid compound

(71) Applicant: Hoffmann-La Roche Inc., Little Falls, NJ (US)

(72) Inventors: Christian Steffen MOESSNER, Lörrach (DE); Helene WOLLEB, Olten (CH)

(73) Assignee: Hoffmann-La Roche Inc., Little Falls, NJ (US)

(21) Appl. No.: 19/423,744

(22) Filed: Dec. 17, 2025

(30) Foreign Application Priority Data
Dec. 18, 2024 (EP) 24221001.1

(51) Int. Cl. C07K 1/107; C07C 231/14

(52) U.S. Cl. CPC C07K 1/1077; C07C 231/14

(57) ABSTRACT

The invention relates to a process for the preparation of a fatty acid compound of the formula Ia

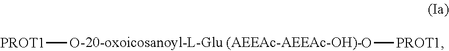

wherein AEEAc stands for 2-(2-(2-aminoethoxy) ethoxy) acetic acid and PROT1 is an ester protecting group and
   to the use of the fatty acid compound of the formula Ia for the preparation of peptides.

### CROSS-REFERENCE TO RELATED APPLICATIONS

This application claims benefit of priority to European Patent Application No. 24221001.1, filed Dec. 18, 2024, which is incorporated herein by reference in its entirety.

### SEQUENCE LISTING

This application contains a Sequence Listing which has been submitted electronically in XML format and is hereby incorporated by reference in its entirety. Said XML copy, created on Feb. 27, 2026, is named P39837-US-1-SequenceListing.xml, and is 4,166 bytes in size.

### TECHNICAL FIELD

The invention relates to a process for the preparation of a fatty acid compound of the formula Ia

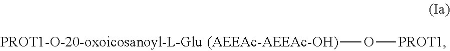

wherein AEEAc stands for 2-(2-(2-aminoethoxy) ethoxy) acetic acid and PROT1 is an ester protecting group and
to the use of the fatty acid compound of the formula Ia, prepared according to the process of the invention, for the preparation of peptides, particularly for the preparation of the peptide of VIIIa (SEQ ID NO:1), or of a pharmaceutically acceptable salt or ester thereof

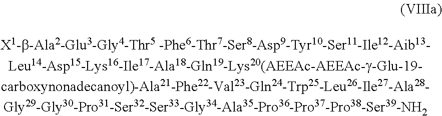

wherein X is

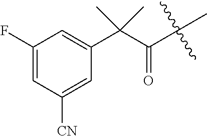 [SMILES: *C(=O)C(C)(C)c1cc(F)cc(C#N)c1]

and
AEEAc stands for 2-(2-(2-aminoethoxy) ethoxy) acetic acid. Likewise the invention relates to a process for the preparation of peptides as outlined above, which make use of the fatty acid compound of the formula I as side chain.

### BACKGROUND

While the fatty acid compounds of the formula Ia are versatile building blocks with a broad scope of potential applications they can find a particular use as side chains in peptides, such as in peptides which have the potential to act as GLP-1R/GIPR agonist as illustrated in the International Patent Publication WO 2022/241287. A particular example is shown with the peptide of formula VIIIa outlined above.

The Chinese Patent Publication CN 117342967 discloses a process for the preparation of peptide building block comprising coupling an amino group protected L-Glu to the N-hydroxysuccimide functionalized dicarboxylic acid ester, functionalizing the free carboxylic acid of L-Glu with N-hydroxysuccinimide and finally coupling AEEAc-AEEAc to the functionalized precursor. The process appears to require additional purification efforts for each reaction step. In addition, the final product has to be further purified by column chromatography, which makes the process not suitable for a process on a larger scale.

### SUMMARY OF INVENTION

The object of the present invention therefore was to find an improved and scalable approach which allows to produce the fatty acid compounds of the formula Ia in high purity and yield and which avoids the disadvantages of the known synthesis.

It was found that the object of the invention could be reached by a process for the preparation of a fatty acid compound of the formula Ia

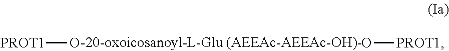

wherein AEEAc stands for 2-(2-(2-aminoethoxy) ethoxy) acetic acid and PROT1 is an ester protecting group,
which comprises the steps:
   a) coupling the eicosanedioic acid ester of formula IIa

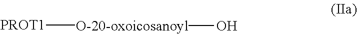

wherein PROT1 is an ester protecting group,
with an amino group protected L-Glu of formula IIIa

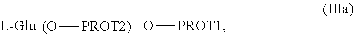

wherein PROT1 and PROT2 are ester protecting groups,
to form the eicosanedioic acid L-Glu ester of formula IVa

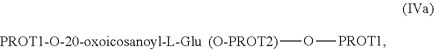

wherein PROT1 and PROT 2 are as above;
b) removing the ester protecting group PROT2 to form the eicosanedioic acid L-Glu acid of formula Va,

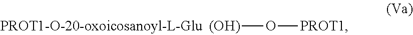

wherein PROT1 is as above;
c) forming the N-hydroxsuccinimide ester of formula VIa,

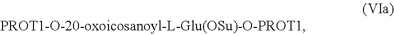

wherein PROT1 is as above
with a N-hydroxysuccinimide source compound; and
d) coupling the N-hydroxsuccinimide ester of formula VIa with 2-(2-{2-[2-(2-amino-ethoxy)-ethoxy]-acetylamino}-ethoxy)-ethoxy]-acetic acid (AEEAc-AEEAc) to form the fatty acid compound of the formula Ia.

2-(2-{2-[2-(2-amino-ethoxy)-ethoxy]-acetylamino}-ethoxy)-ethoxy]-acetic acid (AEEAc-AEEAc) corresponds to 17-amino-10-oxo-3,6,12,15-tetraoxa-9-azaheptadecan-1-oic acid (CAS Reg. No. 1143516 May 5).

### DETAILED DESCRIPTION

The following definitions are set forth to illustrate and define the meaning and scope of the various terms used to describe the invention herein.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcysteine and the like. In addition, these salts may be prepared by addition of an inorganic base or an organic base to the free acid. Salts derived from an inorganic base include but are not limited to, the sodium, potassium, lithium, ammonium, calcium, magnesium salts and the like. Salts derived from organic bases include but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like.

The term “ester protecting group” refers to protecting groups of the carboxylic acid functionalities of the amino acid.

Some ester protecting groups, like tert-butyl (tBu) are cleavable under acidic conditions, e.g. with trifluoroacetic acid while other ester protecting groups, like e.g. benzyl can be removed via catalytic hydrogenolysis. This different cleaving properties can be advantageously applied in the process of the present invention.

The term O-20-oxoicosanoyl refers to the moiety

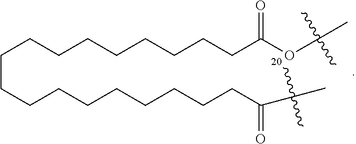 [SMILES: *OC(=O)CCCCCCCCCCCCCCCCCCC(*)=O]

The peptide synthesis is performed in the presence of a coupling agent, which can be selected from benzotriazol-1-yloxytris(dimethylamino)phosphonium hexafluorophosphate (BOP), benzotriazol-1-yloxytripyrrolidinophosphonium hexafluorophosphate (PyBOP), bromotripyrrolidinophosphonium hexafluorophosphate (PyBrOP), (7-azabenzotriazol-1-yloxy)tripyrrolidinophosphonium hexafluorophosphate (PyAOP), N-[(1H-benzotriazol-1-yl) (dimethylamino)methylene]-N-methylmethanaminium tetrafluoroborate N-oxide (TBTU), 2-(1H-benzotriazole-1-yl)-1,1,3,3-tetramethylaminium hexafluorophosphate (HBTU), 1-[Bis(dimethylamino)methylene]-1H-1,2,3-triazolo[4,5-b]pyridinium 3-oxid hexafluorophosphate, N-[(dimethylamino)-3H-1,2,3-triazolo-[4,5-b]pyridin-3-yloxy)methylene]-N-methylmethanaminium hexafluorophosphate (HATU), propanephosphonic acid anhydride (T3P) or from combinations of N,N′-diisopropylcarbodiimide (DIC) with N-hydroxysduccinimide, with 4-(dimethylamino)pyridine (DMAP), with 2-hydroxypyridine-N-oxide (HOPO) or with (ethyl-cyano(hydroximino)acetate) (Oxyma Pure) or from a combination of 1-ethyl-3-(3-dimethylaminopropyl) carbodiimide (EDC) with 2-hydroxypyridine-N-oxide (HOPO).

An organic base, such as a trialkylamine selected from the group consisting of triethylamine, N-methyl morpholine or diisopropylethylamine (DIPEA), preferably diisopropylethylamine (DIPEA), is used for the coupling reactions.

In addition, an organic solvent selected from a polar, aprotic solvent such as N′N-dimethylformamide (DMF), N-alkylpyrrolidones, like N-butyl pyrrolidone, dimethyl sulfoxide (DMSO), ethyl acetate or mixtures thereof, is expediently present.

As outlined above, the process for the preparation of a fatty acid compound of the formula Ia

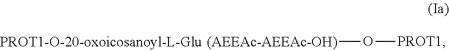

wherein AEEAc stands for 2-(2-(2-aminoethoxy) ethoxy) acetic acid and PROT1 is an ester protecting group,
comprises the steps:
a) coupling the eicosanedioic acid ester of formula Ila

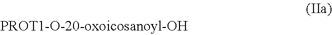

wherein PROT1 is an ester protecting group,
with an amino group protected L-Glu of formula IIIa

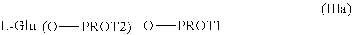

wherein PROT1 and PROT 2 are ester protecting groups,
to form the eicosanedioic acid L-Glu ester of formula IVa

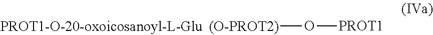

wherein PROT1 and PROT2 are as above;
b) removing the ester protecting group PROT2 in the eicosanedioic acid L-Glu ester of formula IVa to form the eicosanedioic acid L-Glu acid of formula Va,

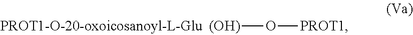

wherein PROT1 is as above;
c) forming the N-hydroxsuccinimide ester of formula VIa,

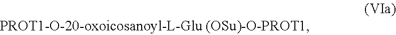

wherein PROT1 is as above
with a N-hydroxysuccinimide source compound; and
d) coupling the N-hydroxsuccinimide ester of formula VIa with 2-(2-{2-[2-(2-amino-ethoxy)-ethoxy]-acetylamino}-ethoxy)-ethoxy]-acetic acid (AEEAc-AEEAc) to form the fatty acid compound of the formula Ia.

In a preferred embodiment, the ester protecting group PROT1 is tert-butyl (tBu) and accordingly the preferred fatty acid compound has the formula Ib.

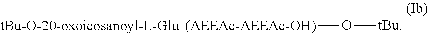

PROT2 is an ester protecting group, which is removable by catalytic hydrogenation, preferably benzyl.

Step a) requires the coupling of the eicosanedioic acid ester of formula Ila with the carboxylic acid protected L-Glu of formula IIIa to form the eicosanedioic acid L-Glu ester of formula IVa.

The coupling in step a) is typically performed in the presence of a coupling agent, an organic base and an organic solvent.

The coupling agent can be selected from benzotriazol-1-yloxytris(dimethylamino)phosphonium hexafluorophosphate (BOP), benzotriazol-1-yloxytripyrrolidinophosphonium hexafluorophosphate (PyBOP), bromotripyrrolidinophosphonium hexafluorophosphate (PyBrOP), (7-azabenzotriazol-1-yloxy)tripyrrolidinophosphonium hexafluorophosphate (PyAOP), N-[(1H-benzotriazol-1-yl) (dimethylamino)methylene]-N-methylmethanaminium tetrafluoroborate N-oxide (TBTU), 2-(1H-benzotriazole-1-yl)-1,1,3,3-tetramethylaminium hexafluorophosphate (HBTU), 1-[Bis(dimethylamino)methylene]-1H-1,2,3-triazolo[4,5-b]pyridinium 3-oxid hexafluorophosphate, N-[(dimethylamino)-3H-1,2,3-triazolo-[4,5-b]pyridin-3-yloxy)methylene]-N-methylmethanaminium hexafluorophosphate (HATU), propanephosphonic acid anhydride (T3P) or from a combinations of N,N′-diisopropylcarbodiimide (DIC) with N-hydroxysduccinimide, with 4-(dimethylamino)pyridine (DMAP), with 2-hydroxypyridine-N-oxide (HOPO) or with (ethyl-cyano(hydroximino)acetate) (Oxyma Pure) or from a combination of 1-ethyl-3-(3-dimethylaminopropyl) carbodiimide (EDC) with 2-hydroxypyridine-N-oxide (HOPO).

Preferred coupling agent is 2-(1H-benzotriazole-1-yl)-1,1,3,3-tetramethylaminium hexafluorophosphate (HBTU).

The organic base is selected from N,N-diisopropylethylamine, N-methylmorpholine (NMM), N-methylimidazol (NMI) or triethylamine, but preferably is N,N-diisopropylethylamine.

The organic solvent is selected from N,N-dimethylformamide, toluene, acetonitrile or mixtures thereof, preferably from a mixture of N,N-dimethylformamide and toluene.

The reaction temperature can be chosen between 10° C. and 30° C., typically the process is run at ambient temperature.

In a preferred aspect of the invention eicosanedioic acid ester of formula IIb

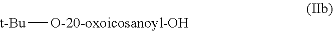

is coupled with an amino group protected L-Glu of formula IIIb

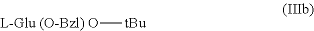

to form the eicosanedioic acid L-Glu ester of formula IVb

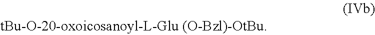

The eicosanedioic acid L-Glu ester of formula IVa or IVb can be isolated from the organic phase by means of standard procedures known to the skilled in the art, however in a more preferred aspect eicosanedioic acid L-Glu ester of formula IVa or IVb is not isolated, but directly, dissolved in the organic solvent, fed to reaction step b).

Step b) requires the removal of the ester protecting group PROT2 in the eicosanedioic acid L-Glu ester of formula IVa and the formation of the eicosanedioic acid L-Glu acid of formula Va.

The removal of the ester protecting group PROT2 is as a rule performed by catalytic hydrogenation with hydrogen and a Pd-catalyst in an organic solvent. Suitable Pd catalysts are Pd 0.5 to 20.0% on carbon and suitable solvents are toluene, tetrahydrofuran or ethylacetate, preferably toluene.

The hydrogenolysis can take place at a temperature of 10° C. to 30° C., preferably at ambient temperature and hydrogen pressures between 1 bar and 5 bar, preferably 1 to 3 bar, more preferably at ambient hydrogen pressure.

In one particular aspect of the invention, the eicosanedioic acid L-Glu (OBzl) ester of formula IVb is transformed to the eicosanedioic acid L-Glu acid of formula Vb,

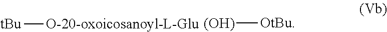

The eicosanedioic acid L-Glu acid of formula Va or Vb can be isolated by filtering off the catalyst, by crystallization from a polar aprotic such as in acetonitrile. Ideally, the eicosanedioic acid L-Glu acid of formula Va or Vb is not isolated, but after filtering off the catalyst and a solvent switch to preferably acetonitrile, directly fed to reaction step c).

Step c) requires the formation of the N-hydroxsuccinimide ester of formula VIa with a N-hydroxysuccinimide source compound.

Suitable N-hydroxysuccinimide source compounds can be selected from N,N′-disuccinimidyl carbonate (DSC), or from N-hydroxysuccinimide in combination with a coupling reagent selected from N,N′-dicyclohexylcarbodiimide (DCC), N,N′-diisopropylcarbodiimide (DIC), 1-ethyl-3-(3-dimethylaminopropyl) carbodiimide (EDC) or 1,1′-carbonyldiimidazol (CDI). N,N′-disuccinimidyl carbonate (DSC) is the preferred N-hydroxysuccinimide source compound.

The reaction is typically performed in the presence of an organic catalyst and an organic solvent at a temperature of 10° C. to 30° C., preferably at ambient temperature.

Suitable organic catalysts are selected from 4-dimethylaminopyridine, N-methylmorpholine, N-methylimidazole, preferably from 4-dimethylaminopyridine.

Suitable organic solvents can be selected from acetonitrile, dichloromethane, N-methyl-2-pyrrolidon (NMP), N,N-dimethylacetamide (DMAC), preferably from acetonitrile.

In a particular aspect of the invention, the N-hydroxysuccinimide ester of formula VIb

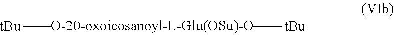

is formed.

The N-hydroxsuccinimide ester of formula VIa or VIb can typically be isolated by filtering off from the reaction mixture and by drying.

Step d) requires the coupling of the N-hydroxsuccinimide ester of formula VIa with 2-(2-{2-[2-(2-amino-ethoxy)-ethoxy]-acetylamino}-ethoxy)-ethoxy]-acetic acid (AEEAc-AEEAc) and the formation of the desired fatty acid compound of the formula Ia.

The reaction is performed in the presence of a silylating agent, an organic base and an organic solvent at a temperature of 10° C. to 30° C., preferably at ambient temperature.

Suitable silylating agents can be selected from trimethylsilylchloride, N,O-bis(trimethylsilyl) acetamide, hexamethyldisilazane N,O-bis(trimethylsilyl)trifluoroacetamide, trimethylsilyl trifluoromethanesulfonate, particularly trimethylsilylchloride.

The organic base is selected from N,N-diisopropylethylamine, triethylamine, N-methylmorpholine, preferably N,N-diisopropylethylamine and the organic solvent is selected from acetonitrile, dichloromethane, N-methyl-2-pyrrolidon (NMP), N,N-dimethylacetamide (DMAC), preferably from acetonitrile.

As outlined above, in a preferred aspect of the invention, the fatty acid compound has the formula Ib:

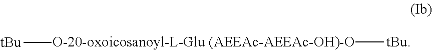

This compound is also named Eic(OtBu)-Glu(AEEAc-AEEAc)-OtBu.

The isolation of the fatty acid compound of formula Ia or Ib can be performed by extraction with an organic solvent, preferably 2-methyltetrahydrofuran, followed by crystallization from a different organic solvent, preferably acetonitrile. Alternatively, the fatty acid can be isolated by evaporation of the solvent and drying.

The process of the present invention allows to obtain the product in a high purity of >99.5% and an overall yield of >80%.

In another aspect of the invention, the fatty acid compound of formula Ia or Ib, prepared in accordance with the process described above can be used for the preparation of peptides, particularly for the preparation of the peptide of formula VIIIa (SEQ ID NO:1), or of a pharmaceutically acceptable salt or ester thereof

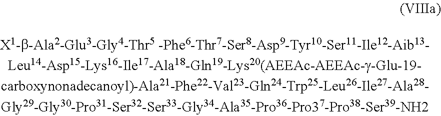

wherein X is

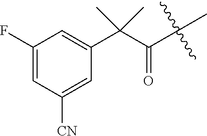 [SMILES: *C(=O)C(C)(C)c1cc(F)cc(C#N)c1]

and
AEEAc stands for 2-(2-(2-aminoethoxy) ethoxy) acetic acid.

In another aspect, the invention comprises a process for the preparation of a peptide comprising the addition of the fatty acid compound of the formula Ia or Ib, prepared according to the process of the preset invention, as a side chain.

In a particular aspect the invention, the peptide has the formula VIIIa (SEQ ID NO:1), or is a pharmaceutically acceptable salt or ester thereof

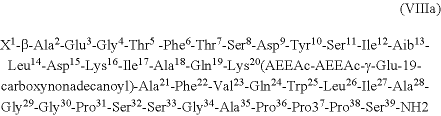

wherein X is

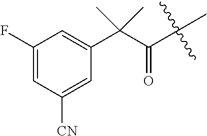 [SMILES: *C(=O)C(C)(C)c1cc(F)cc(C#N)c1]

and
   AEEAc stands for 2-(2-(2-aminoethoxy) ethoxy) acetic acid.

### Examples

Abbreviations:

| | |
|---|---|
| DIPEA | diisopropylethylamine |
| DMAP | 4-dimethylaminopyridine |
| DMF | N,N-dimethylformamide |
| DMSO | dimethylsulfoxide |
| DSC | N,N′-disuccinimidyl carbonate |
| Eic | eicosanedioic acid, or icosanedioic acid |
| EtOAc | ethyl acetate |
| HBTU | hexaafluorophosphate benzotriazole uronium |
| MeCN | acetonitrile |
| 2-MeTHF | 2-methyltetrahydrofuran |
| tBu | tert-butyl |
| TMSCl | trimethylsilylchloride |

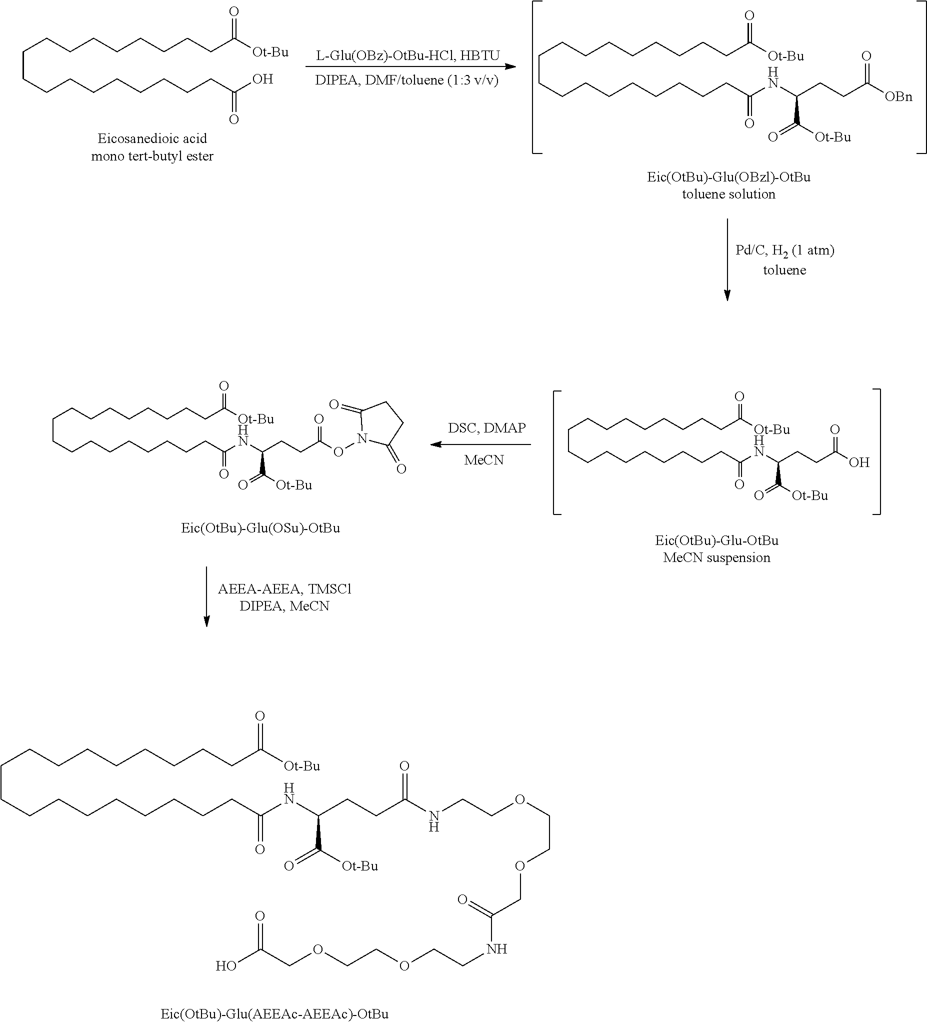

### Synthesis of Eic(OtBu)-Glu(AEEAc-AEEAc)-OtBu (Compound of Formula Ib)

Eicosanedioic acid mono tert-butyl ester (100 g, 251 mmol, 1.00 equiv), L-Glu(OBzl)-OtBu·HCl 5 (88.7 g, 263 mmol, 1.05 equiv) and HBTU (99.9 g, 263 mmol, 1.05 equiv) were taken up in DMF (100 mL, 1 vol.) and toluene (300 mL, 3 vol.) at ambient temperature. DIPEA (81.1 g, 627 mmol, 2.5 equiv) was added over 1 h, and the resulting reaction mixture was stirred at ambient temperature for 4 h. Upon completion of the reaction, toluene (200 mL, 2 vol.) and water (300 mL, 3 vol.) were added and the phases were separated. The organic phase was sequentially washed with 8% NaHCO₃ solution (300 mL+100 mL, 3 vol. +1 vol.) and water (100 mL, 1 vol.) before it was treated with charcoal (2 g, 2 w %) at ambient temperature for 3 h. The resulting mixture was filtered, and the filter cake was washed with toluene (2×100 mL, 2×1 vol.). The resulting filtrate containing Eic(OtBu)-Glu(OBzl)-OtBu was directly used in the following transformation (assumed yield 100%).

¹H NMR (400 MHz, DMSO-d₆): δ 8.05 (d, J=7.6 Hz, 1H), 7.39-7.30 (m, 5H), 5.09 (s, 2H), 4.14 (ddd, J=8.8, 8.0, 1.2 Hz, 1H), 2.46-2.36 (m, 2H), 2.16 (t, J=7.2 Hz, 2H), 2.08 (t, J=7.2 Hz, 2H), 2.01-1.92 (m, 1H), 1.84-1.74 (m, 1H), 1.52-1.42 (m, 4H), 1.39 (s, 9H), 1.38 (s, 9H), 1.22 (s, br, 28H).

5% wet Pd/C (3.38 g, 2 w % with respect to the amount of Eic(OtBu)-Glu(OBzl)-OtBu, water content 50%) was added to the above solution of Eic(OtBu)-Glu(OBzl)-OtBu in toluene (251 mmol, 1.00 equiv) and the resulting reaction mixture was stirred under a H₂ atmosphere at ambient pressure and temperature for 18 h. Upon completion of the reaction, the mixture was filtered, the filter cake was washed with toluene (2×100 mL, 2×0.6 vol.), and the resulting filtrate was concentrated under reduced pressure. The resulting oil was taken up in MeCN (500 mL, 3 vol.) and the mixture was partially concentrated under reduced pressure to afford a suspension. Another portion of MeCN (500 mL, 3 vol.) was added and the suspension was again concentrated under reduced pressure to afford a suspension (ca. 700 g) containing Eic(OtBu)-Glu-OtBu, which was directly used in the following transformation (assumed yield 100%).

¹H NMR (400 MHz, DMSO-d₆): δ 12.14 (s, 1H), 8.04 (d, J=8.0 Hz, 1H), 4.11 (ddd, J=8.8, 8.0, 1.2 Hz, 1H), 2.26 (td, J=8.0, 3.6 Hz, 2H), 2.16 (t, J=7.6 Hz, 2H), 2.09 (t, J=7.6 Hz, 2H), 1.94-1.85 (m, 1H), 1.78-1.68 (m, 1H), 1.52-1.42 (m, 4H), 1.39 (s, br, 18H), 1.23 (s, br, 28H).

DSC (70.7 g, 276 mmol, 1.10 equiv) and the above suspension of Eic(OtBu)-Glu-OtBu in MeCN (251 mmol, 1.00 equiv) were taken up in MeCN (760 mL, 5 vol.), and the resulting suspension was stirred at ambient temperature for 30 min before DMAP (3.1 g, 25 mmol, 0.10 equiv) was added. The resulting reaction mixture was stirred at 30° C. for 1 h. Upon completion of the reaction, the mixture was cooled to 0° C. and was stirred at this temperature for 3 h. The mixture was then warmed to ambient temperature, stirred for 1 h, and cooled again to 0° C. and stirred for 3 h. The resulting suspension was filtered, and the filter cake was washed with pre-cooled (0° C.) MeCN (2×70 mL, 2×0.5 vol.) and dried under vacuum at 25° C. to afford Eic(OtBu)-Glu(OSu)-OtBu (160 g, 90% yield over 3 steps, 96.7% assay purity) as a white solid.

¹H NMR (400 MHz, DMSO-d₆): δ 8.11 (d, J=7.6 Hz, 1H), 4.16 (ddd, J=9.2, 7.6, 1.6 Hz, 1H), 2.81 (s, 4H), 2.79-2.63 (m, 2H), 2.16 (t, J=6.0 Hz, 2H), 2.11 (t, J=6.0 Hz, 2H), 2.06-1.99 (m, 1H), 1.93-1.83 (m, 1H), 1.50-1.45 (m, 4H), 1.39 (s, 9H), 1.38 (s, 9H), 1.23 (s, br, 28H).

TMSCl (10.4 g, 95.7 mmol, 1.30 equiv) was taken up in MeCN (258 mL, 5 vol.). AEEAc-AEEAc (25.0 g, 81.1 mmol, 1.10 equiv) was added in one portion at ambient temperature, and the resulting suspension was stirred at this temperature for 1 h to afford a clear solution. A suspension of Eic(OtBu)-Glu(OSu)-OtBu (52.0 g, 73.8 mmol, 1.00 equiv) in MeCN (258 mL, 5 vol.) was added, followed by DIPEA (23.9 g, 185 mmol, 2.50 equiv). The resulting reaction mixture was stirred at ambient temperature for 2 h. Upon completion of the reaction, water (2 mL) was added, and the mixture was stirred at ambient temperature for 10 min before 10% aq. HCl (2.7 mL) was added and the mixture was concentrated under reduced pressure. The resulting oil was taken up in 2-MeTHF (260 mL, 5 vol.) and water (156 mL, 3 vol.), and 10% aq. HCl (43 mL, 0.8 vol.) was added. The phases were separated, and the organic phase was washed with 2% aq. NaCl solution (6×156 mL, 6×3 vol.) and concentrated under reduced pressure. The resulting residue was taken up in MeCN (260 mL, 5 vol.), and the resulting mixture was again concentrated under reduced pressure. This step was repeated once again. Then, the residue was taken up in MeCN (520 mL, 10 vol.), and the mixture was cooled to −15° C. The resulting suspension was stirred at this temperature for 3 h before it was filtered and the filter cake was washed with pre-cooled (−10° C.) MeCN (50 mL, 1 vol.). The resulting solid was dried under vacuum while gradually increasing the temperature from −10° C. to 20° C. to afford Eic(OtBu)-Glu(AEEAc-AEEAc)-OtBu (58 g, 90% yield, purity 99.7 area %) as a white solid.

¹H NMR (400 MHz, DMSO-d₆): δ 8.05 (d, J=7.5 Hz, 1H), 7.89 (t, J=5.5 Hz, 1H), 7.65 (t, J=5.7 Hz, 1H), 4.07-4.02 (m, 1H), 4.00 (s, 2H), 3.87 (s, 2H), 3.59-3.51 (m, 8H), 3.44-3.34 (m, 5H), 3.27 (q, J=5.8 Hz, 2H), 3.20 (q, J=5.8 Hz, 2H), 2.17-2.07 (m, 6H), 1.93-1.84 (m, 1H), 1.78-1.68 (m, 1H), 1.47 (s, br, 4H), 1.37 (s, br, 18H), 1.23 (s, br, 28H).

1. A process for the preparation of a fatty acid compound of formula Ia 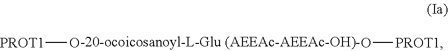
wherein AEEAc stands for 2-(2-(2-aminoethoxy) ethoxy) acetic acid and PROT1 is an ester protecting group,
comprising the steps;
a) coupling the eicosanedioic acid ester of formula IIa 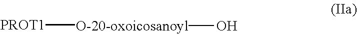
wherein PROT1 is an ester protecting group,
with a carboxyl group protected L-Glu of formula IIIa 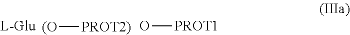
wherein PROT1 and PROT2 are ester protecting groups,
to form the eicosanedioic acid L-Glu ester of formula IVa 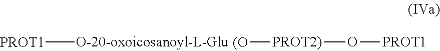
wherein PROT1 and PROT2 are as above;
b) removing the ester protecting group PROT2 to form the eicosanedioic acid L-Glu acid of formula Va, 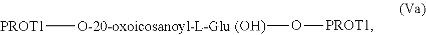
wherein PROT1 is as above;
c) forming the N-hydroxsuccinimide ester of formula VIa, 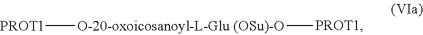
wherein PROT1 is as above
with a N-hydroxysuccinimide source compound; and
d) coupling the N-hydroxsuccinimide ester of formula VIa with 2-(2-{2-[2-(2-amino-ethoxy)-ethoxy]-acetylamino}-ethoxy)-ethoxy]-acetic acid (AEEAc-AEEAc) to form the fatty acid compound of formula Ia.

2. The process of claim 1, wherein PROT1 is tert-butyl (tBu).

3. The process of claim 1, wherein PROT2 is an ester protecting group which is removable by catalytic hydrogenation.

4. The process of claim 3, wherein PROT2 is benzyl.

5. The process of claim 1, wherein the coupling in step a) is performed in the presence of a coupling agent, an organic base and an organic solvent.

6. The process of claim 5, wherein the coupling agent is selected from benzotriazol-1-yloxytris(dimethylamino)phosphonium hexafluorophosphate (BOP), benzotriazol-1-yloxytripyrrolidinophosphonium hexafluorophosphate (PyBOP), bromotripyrrolidinophosphonium hexafluorophosphate (PyBrOP), (7-azabenzotriazol-1-yloxy)tripyrrolidinophosphonium hexafluorophosphate (PyAOP), N-[(1H-benzotriazol-1-yl) (dimethylamino)methylene]-N-methylmethanaminium tetrafluoroborate N-oxide (TBTU), 2-(1H-benzotriazole-1-yl)-1,1,3,3-tetramethylaminium hexafluorophosphate (HBTU), 1-[Bis(dimethylamino)methylene]-1H-1,2,3-triazolo[4,5-b]pyridinium 3-oxid hexafluorophosphate, N-[(dimethylamino)-3H-1,2,3-triazolo-[4,5-b]pyridin-3-yloxy)methylene]-N-methylmethanaminium hexafluorophosphate (HATU), propanephosphonic acid anhydride (T3P) or from a combination of N,N′-diisopropylcarbodiimide (DIC) with N-hydroxysduccinimide, with 4-(dimethylamino)pyridine (DMAP), with 2-hydroxypyridine-N-oxide (HOPO) or with (ethyl-cyano(hydroximino)acetate) or from a combination of 1-ethyl-3-(3-dimethylaminopropyl) carbodiimide (EDC) with 2-hydroxypyridine-N-oxide (HOPO).

7. The process of claim 5, wherein the organic base is selected from N,N-diisopropylethylamine, N-methylmorpholine (NMM), N-methylimidazol (NMI) and triethylamine.

8. The process of claim 5, wherein the organic solvent is selected from N,N-dimethylformamide, toluene, acetonitrile and mixtures thereof.

9. The process of claim 1, wherein the eicosanedioic acid L-Glu ester of formula IVa is not isolated, but directly fed to reaction step b).

10. The process of claim 1, wherein the removal of the ester protecting group PROT2 in step b) is performed by catalytic hydrogenation with hydrogen and a Pd-catalyst in an organic solvent.

11. The process of claim 1, wherein the eicosanedioic acid L-Glu acid of formula Va is not isolated, but after filtering-off of the catalyst directly fed to reaction step c).

12. The process of claim 1, wherein the N-hydroxysuccinimide source compound applied for the formation of the N-hydroxsuccinimide ester of formula VIa in step c) is selected from N,N′-disuccinimidyl carbonate (DSC), or from N-hydroxysuccinimide in combination with a coupling reagent selected from N,N′-dicyclohexylcarbodiimide (DCC), N,N′-diisopropylcarbodiimide (DIC), 1-ethyl-3-(3-dimethylaminopropyl) carbodiimide (EDC) or 1,1′-carbonyldiimidazol (CDI).

13. The process of claim 1, wherein the formation of the N-hydroxsuccinimide ester of formula VIa in step c) is performed in the presence of an organic catalyst and an organic solvent.

14. The process of claim 13, wherein the organic catalyst is selected from 4-dimethylaminopyridine, N-methylmorpholine and N-methylimidazole.

15. The process of claim 13, wherein the organic solvent is selected from acetonitrile, dichloromethane, N-methyl-2-pyrrolidon (NMP) and N,N-dimethylacetamide (DMAC).

16. The process of claim 1, wherein the coupling of the N-hydroxsuccinimide ester of formula VIa with 2-(2-{2-[2-(2-amino-ethoxy)-ethoxy]-acetylamino}-ethoxy)-ethoxy]-acetic acid (AEEAc-AEEAc) is performed in the presence of a silylating agent, an organic base and an organic solvent.

17. The process of claim 16, wherein the silylating agent is selected from trimethylsilylchloride, N,O-bis(trimethylsilyl) acetamide, hexamethyldisilazane, N,O-bis(trimethylsilyl)-trifluoroacetamide and trimethylsilyl trifluoromethanesulfonate.

18. The process of claim 16, wherein the organic base is selected from N,N-diisopropylethylamine, triethylamine and N-methylmorpholine.

19. The process of claim 16, wherein the organic solvent is selected from acetonitrile, dichloromethane, N-methyl-2-pyrrolidon (NMP) and N,N-dimethylacetamide (DMAC).

20. A method of using the fatty acid compound of formula Ia, prepared according to claim 1, for the preparation of peptides.

21. The method of claim 20 for the preparation of the peptide of formula VIIIa, or of a pharmaceutically acceptable salt or ester thereof 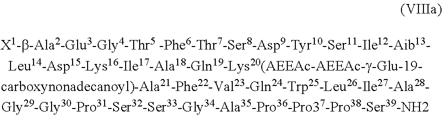
wherein X is 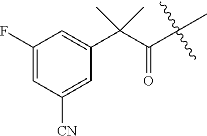 [SMILES: *C(=O)C(C)(C)c1cc(F)cc(C#N)c1]
and
AEEAc stands for 2-(2-(2-aminoethoxy) ethoxy) acetic acid.

22. A process for the preparation of a peptide comprising the addition of the fatty acid compound of formula Ia, prepared according to claim 1, as side chain.

23. The process of claim 22, wherein the peptide has formula VIIIa, or of a pharmaceutically acceptable salt or ester thereof 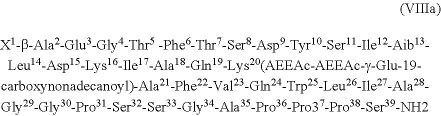
wherein X is 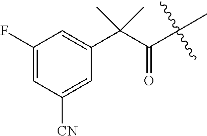 [SMILES: *C(=O)C(C)(C)c1cc(F)cc(C#N)c1]
and
AEEAc stands for 2-(2-(2-aminoethoxy) ethoxy) acetic acid.


SEQUENCE LISTING (1 sequence)

SEQ ID NO: 1 - protein, 38 aa, synthetic construct
  MOD_RES 38: note = S at position 38: the C-terminal amino acid is amidated as a C-terminal primary amide
  SITE 1: note = N-[2-(3-cyano-5-fluorophenyl)-2-methyl-1-oxopropyl] is attached to the amino acid at position 1
  SITE 1: note = X at position 1 corresponds to beta-alanyl
  SITE 12: note = X at position 12 corresponds to Aib
  SITE 19: note = X at position 19 corresponds to N6-[N-(19-carboxy-1 -oxononadecyl)-L-gamma-glutamyl-2-[2-(2-aminoethoxy)ethoxy ]acetyl-2-[2-(2-aminoethoxy)ethoxy]acetyl]-L-lysyl
XEGTFTSDYSIXLDKIAQXAFVQWLIAGGPSSGAPPPS